(19) United States
(12) Patent Application Publication
(10) Pub. No.: US 2011/0178311 A1
(43) Pub. Date: Jul. 21, 2011

(54) Purification of Progesterone Receptor Modulators

(75) Inventors: Mahmut Levent, Bloomfield, NJ (US); Panolil Raveendranath, Monroe, NY (US); Bogdan Wilk, New City, NY (US); Donald Carl Boop, Bloomsbury, NJ (US); Marinus Cornelis Hofman, Nanuet, NY (US)

(73) Assignee: Wyeth LLC, Madison, NJ (US)

(21) Appl. No.: 13/078,273

(22) Filed: Apr. 1, 2011

Related U.S. Application Data
(63) Continuation of application No. 11/713,971, filed on Mar. 5, 2007, now abandoned.
(60) Provisional application No. 60/779,938, filed on Mar. 7, 2006.

(51) Int. Cl. C07D 403/10

(52) U.S. Cl. 548/411

(57) ABSTRACT

Processes are provided for purifying a compound of the structure (I):

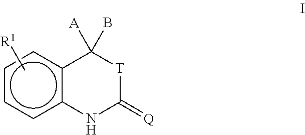

wherein, A, B, T, Q and R¹ are defined herein, and wherein the process includes dissolving the compound of formula I in a solution containing acetone, water and a base at about 30° C.; filtering the solution containing the compound of formula I at about 30° C.; and precipitating the purified compound of formula I by adjusting the filtered solution to an acidic pH. Desirably, the compound of formula I is 5-(2-thioxospiro[cyclohexane-1,3-[3H]indol]-5-yl)-1-methylpyrrole-2-carbonitrile or tanaproget.

### CROSS-REFERENCE TO RELATED APPLICATIONS

This application is a continuation of U.S. patent application Ser. No. 11/713,971, filed Mar. 5, 2007, which claims the benefit of the priority of U.S. Provisional Patent Application No. 60/779,938, filed Mar. 7, 2006, now expired. These priority applications are herein incorporated by reference.

### BACKGROUND OF THE INVENTION

This invention relates to processes for purifying progesterone receptor modulators.

The natural hormone, or ligand, for the progesterone receptor (PR) is the steroid progesterone, but synthetic compounds, such as medroxyprogesterone acetate or levonorgestrel, have been made which also serve as PR ligands. Once a ligand is present in the fluid surrounding a cell, it passes through the membrane via passive diffusion, and binds to the PR to create a receptor/ligand complex. This complex binds to specific gene promoters present in the cell's DNA. Once bound to the DNA, the complex modulates the production of mRNA and the protein encoded by that gene.

A compound that binds to a PR and mimics the action of the natural hormone is termed a PR agonist, whilst a compound which inhibits the effect of the hormone is a PR antagonist. PR agonists and antagonists (natural and synthetic) are known to play an important role in the health of women. PR agonists and antagonists are used in birth control formulations, either alone or in the presence of another active agent. The PR modulators may also be useful for the treatment of hormone dependent breast cancers, for the treatment of non-malignant chronic conditions such as uterine fibroids, and endometriosis.

Methods of purifying progesterone receptor modulators have been described previously. For example, US Patent Application Publication No. US-2005-0250766 describes a process for treating a crude form of a PR modulator, 5-(2-thioxospiro[cyclohexane-1,3-[3H]indol]-5-yl)-1-methylpyrrole-2-carbonitrile, with a solvent to form a basic salt and converting the basic salt to a purified PR modulator via a solubilizing salt. Other processes for purifying PR modulators have been been described and include purification via a classical crystallization from acetone-water after a continuous filtration of the acetone solution through a charcoal pad at 48° C. The acetone typically is then removed by distillation and, after the addition of water and cooling, crystals of the product are formed and thereby isolated by filtration. One disadvantage to this process is low yield of the purified product.

Alternate processes for purifying progesterone receptor modulators are needed.

### SUMMARY OF THE INVENTION

In one aspect, processes are provided for purifying progesterone receptor modulators.

In another aspect, processes for purifying a compound of formula I, wherein A, B, T, Q, and R¹ are defined below, are described. The processes include a hydroxide base and temperatures of about 30° C.

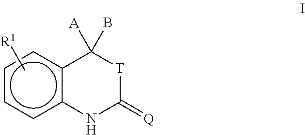

In a further aspect, a process is provided for purifying 5-(2-thioxospiro[cyclohexane-1,3-[3H]indol]-5-yl)-1-methylpyrrole-2-carbonitrile.

In yet another aspect, a process for purifying tanaproget is described.

Other aspects and advantages of the invention will be readily apparent from the following detailed description of the invention.

### DETAILED DESCRIPTION OF THE INVENTION

A process is provided for purifying progesterone receptor modulator compounds. The inventors found that the process described herein, which utilizes a base/acetone/water solution at a temperature of at least about 20° C., and preferably about 30° C., avoids side reactions. More particularly, the inventors found that higher temperatures generated impurities and that lower temperatures do not permit dissolution of the crude compound in the solvent. Thus, the processes for purifying progesterone receptor modulators as provided herein provide high yields of the purified compound. Further, the purification processes herein do not require any distilling steps and avoid premature precipitation of the purified compound, including premature precipitation during the filtration steps.

Without wishing to be bound by theory, the inventors believe that the low yields of purified progesterone receptor modulators, when purified using the classical (prior art) method, is caused by several factors. First, the 5-(2-thioxospiro[cyclohexane-1,3-[3H]indol]-5-yl)-1-methylpyrrole-2-carbonitrile progesterone receptor modulator has a low solubility in the acetone solvent, which thereby requires the use of a large amount of acetone on a small scale and even larger amounts during scale-up. Second, filtration of the acetone/water solution utilized in the purification through activated carbon was difficult due to the decreased solubility of the 5-(2-thioxospiro[cyclohexane-1,3-[3H]indol]-5-yl)-1-methylpyrrole-2-carbonitrile in the acetone. Finally, once the filtration step was completed, it was necessary to isolate the purified product by distilling the large amounts of acetone to provide the purified product.

The processes described herein thereby avoid any need to utilize large amounts of solvents that do not efficiently dissolve the compound being purified, which solvents are difficult to remove after the purification. Therefore, the requirement to utilize solvents and solvent systems which include tetrahydrofuran, and mixtures containing same is eliminated.

### I. Definitions

The term “alkyl” is used herein to refer to both straight- and branched-chain saturated aliphatic hydrocarbon groups having one to ten carbon atoms, such as one to eight carbon atoms, one to six carbon atoms, or one or four carbon atoms. The term “lower alkyl” refers to straight- and branched-chain saturated aliphatic hydrocarbon groups having one to six carbon atoms. The term “alkenyl” refers to both straight- and branched-chain alkyl groups with at least one carbon-carbon double bond and two to eight carbon atoms, two to six carbon atoms, or two to four carbon atoms. The term “alkynyl refers to both straight- and branched-chain alkyl groups with at least one carbon-carbon triple bond and two to eight carbon atoms, two to six carbon atoms, or two to four carbon atoms.

The terms “substituted alkyl”, “substituted alkenyl”, and “substituted alkynyl” refer to alkyl, alkenyl, and alkynyl groups as just described having from one to three substituents including halogen, CN, OH, NO₂, amino, aryl, substituted aryl, heterocyclic, substituted heterocyclic, heteroaryl, substituted heteroaryl, alkoxy, substituted alkoxy, aryloxy, substituted aryloxy, alkylcarbonyl, alkylcarboxy, alkylamino, and arylthio. These substituents may be attached to any carbon of an alkyl, alkenyl, or alkynyl group provided that the attachment constitutes a stable chemical moiety.

The term “cycloalkyl” is used herein to describe a carbon-based saturated ring having more than 3 carbon-atoms and which forms a stable ring. The term cycloalkyl can include groups where two or more cycloalkyl groups have been fused to form a stable multicyclic ring. Desirably, cycloalkyl refers to a ring having about 4 to about 9 carbon atoms, and more desirably about 6 carbon atoms.

The term “substituted cycloalkyl” is used herein to refer to a cycloalkyl group as just described and having from one to five substituents including, without limitation, hydrogen, halogen, CN, OH, NO₂, amino, alkyl, substituted alkyl, alkenyl, substituted alkenyl, alkynyl, alkoxy, aryloxy, substituted alkyloxy, alkylcarbonyl, alkylcarboxy, alkylamino, substituted alkylamino, arylthio, heterocyclic, substituted heterocyclic, heteroaryl, substituted heteroaryl, aminoalkyl, and substituted aminoalkyl.

The term “aryl” is used herein to refer to a carbocyclic aromatic system, which may be a single ring, or multiple aromatic rings fused or linked together as such that at least one part of the fused or linked rings forms the conjugated aromatic system. The aryl groups include, but are not limited to, phenyl, naphthyl, biphenyl, anthryl, tetrahydronaphthyl, phenanthryl, and indane.

The term “substituted aryl” refers to aryl as just defined having one to four substituents including halogen, CN, OH, NO₂, amino, alkyl, cycloalkyl, alkenyl, alkynyl, alkoxy, aryloxy, substituted alkyloxy, alkylcarbonyl, alkylcarboxy, alkylamino, and arylthio.

The term “heterocycle” or “heterocyclic” as used herein can be used interchangeably to refer to a stable, saturated or partially unsaturated 3- to 9-membered monocyclic or multicyclic heterocyclic ring. The heterocyclic ring has in its backbone carbon atoms and one or more heteroatoms including nitrogen, oxygen, and sulfur atoms. In one embodiment, the heterocyclic ring 1 to about 4 heteroatoms in the backbone of the ring. When the heterocyclic ring contains nitrogen or sulfur atoms in the backbone of the ring, the nitrogen or sulfur atoms can be oxidized. The term “heterocycle” or “heterocyclic” also refers to multicyclic rings in which a heterocyclic ring is fused to an aryl ring of about 6 to about 14 carbon atoms. The heterocyclic ring can be attached to the aryl ring through a heteroatom or carbon atom provided the resultant heterocyclic ring structure is chemically stable. In one embodiment, the heterocyclic ring includes multicyclic systems having 1 to 5 rings.

A variety of heterocyclic groups are known in the art and include, without limitation, oxygen-containing rings, nitrogen-containing rings, sulfur-containing rings, mixed heteroatom-containing rings, fused heteroatom containing rings, and combinations thereof. Examples of heterocyclic groups include, without limitation, tetrahydrofuranyl, piperidinyl, 2-oxopiperidinyl, pyrrolidinyl, morpholinyl, thiamorpholinyl, thiamorpholinyl sulfoxide, pyranyl, pyronyl, dioxinyl, piperazinyl, dithiolyl, oxathiolyl, dioxazolyl, oxathiazolyl, oxazinyl, oxathiazinyl, benzopyranyl, benzoxazinyl and xanthenyl.

The term “heteroaryl” as used herein refers to a stable, aromatic 5- to 14-membered monocyclic or multicyclic heteroatom-containing ring. The heteroaryl ring has in its backbone carbon atoms and one or more heteroatoms including nitrogen, oxygen, and sulfur atoms. In one embodiment, the heteroaryl ring contains 1 to about 4 heteroatoms in the backbone of the ring. When the heteroaryl ring contains nitrogen or sulfur atoms in the backbone of the ring, the nitrogen or sulfur atoms can be oxidized. The term “heteroaryl” also refers to multicyclic rings in which a heteroaryl ring is fused to an aryl ring. The heteroaryl ring can be attached to the aryl ring through a heteroatom or carbon atom provided the resultant heterocyclic ring structure is chemically stable. In one embodiment, the heteroaryl ring includes multicyclic systems having 1 to 5 rings.

A variety of heteroaryl groups are known in the art and include, without limitation, oxygen-containing rings, nitrogen-containing rings, sulfur-containing rings, mixed heteroatom-containing rings, fused heteroatom containing rings, and combinations thereof. Examples of heteroaryl groups include, without limitation, furyl, pyrrolyl, pyrazolyl, imidazolyl, triazolyl, pyridyl, pyridazinyl, pyrimidinyl, pyrazinyl, triazinyl, azepinyl, thienyl, dithiolyl, oxathiolyl, oxazolyl, thiazolyl, oxadiazolyl, oxatriazolyl, oxepinyl, thiepinyl, diazepinyl, benzofuranyl, thionapthene, indolyl, benzazolyl, purindinyl, pyranopyrrolyl, isoindazolyl, indoxazinyl, benzoxazolyl, quinolinyl, isoquinolinyl, benzodiazonyl, napthylridinyl, benzothienyl, pyridopyridinyl, acridinyl, carbazolyl, and purinyl rings.

The term “substituted heterocycle” and “substituted heteroaryl” as used herein refers to a heterocycle or heteroaryl group having one or more substituents including halogen, CN, OH, NO₂, amino, alkyl, cycloalkyl, alkenyl, alkynyl, C₁ to C₃ perfluoroalkyl, C₁ to C₃ perfluoroalkoxy, alkoxy, aryloxy, alkyloxy including —O—(C₁ to C₁₀ alkyl) or —O—(C₁ to C₁₀ substituted alkyl), alkylcarbonyl including —CO—(C₁ to C₁₀ alkyl) or —CO—(C₁ to C₁₀ substituted alkyl), alkylcarboxy including —COO—(C₁ to C₁₀ alkyl) or —COO—(C₁ to C₁₀ substituted alkyl), —C(NH₂)═N—OH, —SO₂—(C₁ to C₁₀ alkyl), —SO₂—(C₁ to C₁₀ substituted alkyl), —O—CH₂-aryl, alkylamino, arylthio, aryl, substituted aryl, heteroaryl, or substituted heteroaryl which groups may be optionally substituted. A substituted heterocycle or heteroaryl group may have 1, 2, 3, or 4 substituents.

The term “alkoxy” is used herein to refer to the OR group, where R is alkyl or substituted alkyl. The term “lower alkoxy” refers alkoxy groups having one to six carbon atoms.

The term “aryloxy” is used herein to refer to the OR group, where R is aryl or substituted aryl.

The term “arylthio” is used herein to refer to the SR group, where R is aryl or substituted aryl.

The term “alkylcarbonyl” is used herein to refer to the RCO group, where R is alkyl or substituted alkyl.

The term “alkylcarboxy” is used herein to refer to the COOR group, where R is alkyl or substituted alkyl.

The term “aminoalkyl” refers to both secondary and tertiary amines wherein the alkyl or substituted alkyl groups, containing one to eight carbon atoms, which may be either same or different and the point of attachment is on the nitrogen atom.

The term “halogen” refers to Cl, Br, F, or I.

The term “ester” as used herein refers to a C(O)O, where the points of attachment are through both the carbon and oxygen atoms. One or both oxygen atoms of the ester group can be replaced with a sulfur atom, thereby forming a “thioester”, i.e., a C(O)S, C(S)O or C(S)S group.

The term “purified” or “pure” as used herein refers to a compound that contains less than about 10% impurities. In one example, the term “purified” or “pure” refers to a compound that contains less than about 5% impurities. In another example, the term “purified” or “pure” refers to a compound that contains less than about 2% impurities. In a further example, the term “purified” or “pure” refers to a compound that contains less than about 1% impurities. The term “purified” or “pure” can also refer to a compound that contains about 0% impurities.

The term “crude” as used herein refers to a compound that contains greater than about 10% impurities. In one example, the term “crude” refers to a compound that contains greater than about 5% impurities. In another example, the term “crude” refers to a compound that contains greater than about 2% impurities. In a further example, the term “crude” refers to a compound that contains greater than about 1% impurities. The impurities that can be present in a crude sample can include unused starting materials or undesirable side products formed during the reaction to form the crude compound. In one embodiment, such impurities are present as solids. The impurities can also include solvents that are present or trapped in the crude compound.

By the term “dry” or “drying” is meant a procedure by which entrapped solvents, including solvents, or water, or volatile solids are removed from a sample.

The term “electron withdrawing group” as used herein is meant to describe a chemical substituent that withdraws electrons from the chemical group to which it is attached. Examples of electron withdrawing groups include, without limitation, CN, SO₃H, CO₂H, CO₂R, CHO, COR, NO₂, NR₃⁺, CF₃, or CCl₃. In one embodiment, the electron withdrawing group is CN.

### II. The Purification Process

Processes are provided for purifying a variety of progesterone receptor modulators. In one embodiment, processes are provided for purifying indolone, indol-thione, indol-ylidene cyanamide, benzoxazinone, benzoxazin-thione, benzoxazin-ylidene cyanamide, benzothiazinone, benzothiazine-thione, benzothiazin-ylidene cyanamide compounds, or derivatives thereof. In a further embodiment, purified indol-2-one, indol-2-thione, indol-2-ylidene cyanamide, benzoxazin-2-one, benzoxazin-2-thione, benzoxazin-2-ylidene cyanamide, benzothiazin-2-one, benzothiazine-2-thione, benzothiazin-2-ylidene cyanamide compounds, or derivatives thereof, are prepared according to the processes described herein.

In another embodiment, processes are described for purifying compounds of formula I.

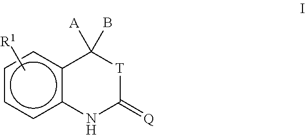

wherein, A and B are independently selected from H, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₃ to C₈ cycloalkyl, substituted C₃ to C₈ cycloalkyl, aryl, substituted aryl, heterocyclic, substituted heterocyclic, COR^{A}, or NR^{B}COR^{A}. Alternatively, A and B are joined to form a ring including (i) a carbon-based 3 to 8 membered saturated spirocyclic ring; (ii) a carbon-based 3 to 8 membered spirocyclic ring containing in its backbone one or more carbon-carbon double bonds; or (iii) a 3 to 8 membered heterocyclic ring containing in its backbone one to three heteroatoms selected from among O, S and N. The rings are optionally substituted by from 1 to 4 groups independently selected from among fluorine, C₁ to C₆ alkyl, C₁ to C₆ alkoxy, C₁ to C₆ thioalkyl, CF₃, OH, CN, NH₂, NH(C₁ to C₆ alkyl), or N(C₁ to C₆ alkyl)₂. In one embodiment, A and B are independently C₁ to C₆ alkyl or are fused to form a carbon-based saturated spirocyclic ring. R^{A} is selected from among H, C₁ to C₃ alkyl, substituted C₁ to C₃ alkyl, aryl, substituted aryl, C₁ to C₃ alkoxy, substituted C₁ to C₃ alkoxy, amino, C₁ to C₃ aminoalkyl, or substituted C₁ to C₃ aminoalkyl. R^{B} is selected from among H, C₁ to C₃ alkyl, or substituted C₁ to C₃ alkyl. T is selected from among O, S, or is absent and Q is selected from among O, S, or NR³. R³ may be an electron withdrawing group. In one example, R³ is selected from among C₁ to C₆ alkyl, substituted C₁ to C₆ alkyl, aryl, substituted aryl, CN, C(O)R⁴, SO₂R⁴, SCN, OR⁴, SR⁴, C(O)OR⁴, C(S)OR⁴, C(O)SR⁴, or C(S)SR⁴ and R⁴ is selected from among C₁ to C₆ alkyl, substituted C₁ to C₆ alkyl, aryl, or substituted aryl. In another example, R³ is CN.

R¹ is located at any position on the ring. In one embodiment, R¹ is halogen. In another embodiment, R¹ is bromine. In still another embodiment, R¹ is a substituted benzene ring containing the substituents X, Y and Z as shown below:

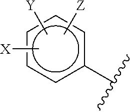

wherein, X is selected from among H, halogen, CN, C₁ to C₃ alkyl, substituted C₁ to C₃ alkyl, alkenyl, substituted alkenyl, alkynyl, substituted alkynyl, C₁ to C₃ alkoxy, substituted C₁ to C₃ alkoxy, C₁ to C₃ thioalkoxy, substituted C₁ to C₃ thioalkoxy, amino, C₁ to C₃ aminoalkyl, substituted C₁ to C₃ aminoalkyl, NO₂, C₁ to C₃ perfluoroalkyl, 5 or 6 membered heterocyclic ring containing in its backbone 1 to 3 heteroatoms, SO₂NH₂, COR^{C}, OCOR^{C}, or NR^{D}COR^{C}; R^{C} is selected from among H, C₁ to C₃ alkyl, substituted C₁ to C₃ alkyl, aryl, substituted aryl, C₁ to C₃ alkoxy, substituted C₁ to C₃ alkoxy, C₁ to C₃ aminoalkyl, or substituted C₁ to C₃ aminoalkyl; R^{D} is selected from among H, C₁ to C₃ alkyl, or substituted C₁ to C₃ alkyl; and Y and Z are independently selected from among H, halogen, CN, NO₂, amino, aminoalkyl, C₁ to C₃ alkoxy, C₁ to C₃ alkyl, or C₁ to C₃ thioalkoxy.

In another embodiment, R¹ is a five or six membered heterocyclic ring containing 1, 2, or 3 heteroatoms or heteroatom containing groups including O, S, SO, SO₂ or NR² and containing one or two substituents independently selected from among H, halogen, CN, NO₂, amino, C₁ to C₃ alkyl, C₁ to C₃ alkoxy, C₁ to C₃ aminoalkyl, SO₂NH₂, COR^{E}, or NR^{F}COR^{E}; R^{E} is selected from among H, C₁ to C₃ alkyl, substituted C₁ to C₃ alkyl, aryl, substituted aryl, C₁ to C₃ alkoxy, substituted C₁ to C₃ alkoxy, C₁ to C₃ aminoalkyl, or substituted C₁ to C₃ aminoalkyl; and R² is selected from among H, C₁ to C₃ alkyl, or substituted C₁ to C₃ alkyl. R² is absent or selected from among H, O, or C₁ to C₄ alkyl. In one embodiment, R¹ is a pyrrole ring, or a pyrrole ring having a cyano substituent.

In another embodiment, the following compounds are purified as described herein, where A, B, and R¹ are as defined above.

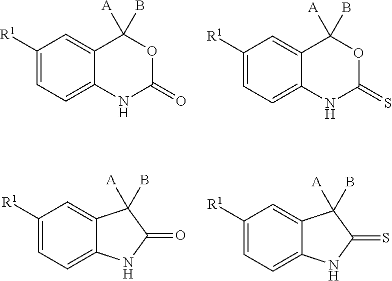

In a further embodiment, the following compounds are purified.

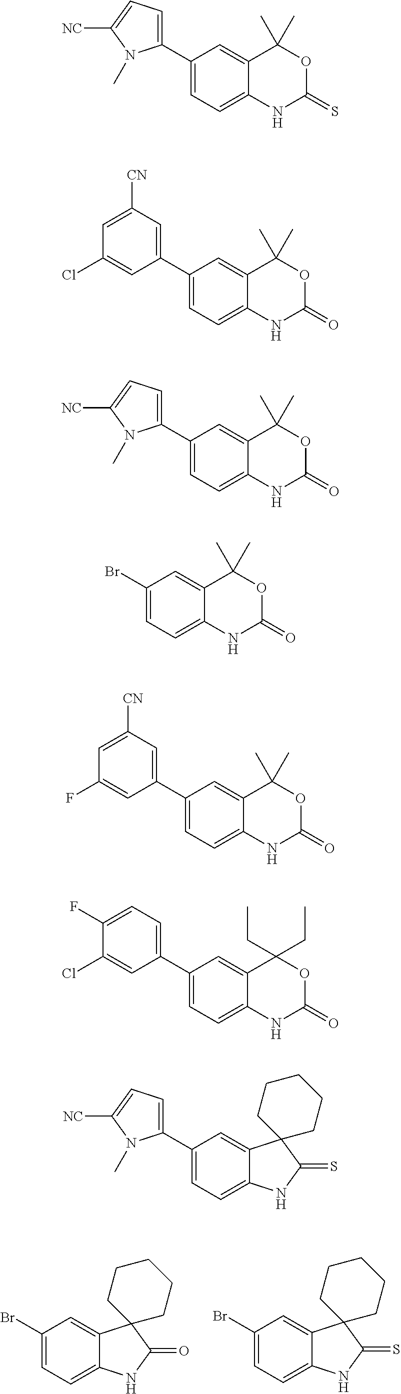

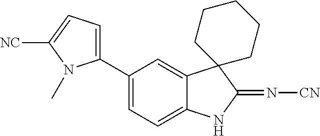

In another embodiment, the compounds produced according to the methods described in U.S. Pat. Nos. 6,509,334; 6,566,358; 6,391,907; 6,608,068; 6,466,648; 6,521,657; 6,583,145; 6,436,929; 6,407,101; 6,562,857; 5,171,851; and 5,874,430; and Singh (J. Med. Chem., 37:248-254 (1994)) are purified according to the processes herein.

In yet a further embodiment, 5-(2-thioxospiro[cyclohexane-1,3-[3H]indol]-5-yl)-1-methylpyrrole-2-carbonitrile and tanaproget are purified according to the processes herein.

Processes are thereby provided for purifying a compound of formula I. See, Scheme 1.

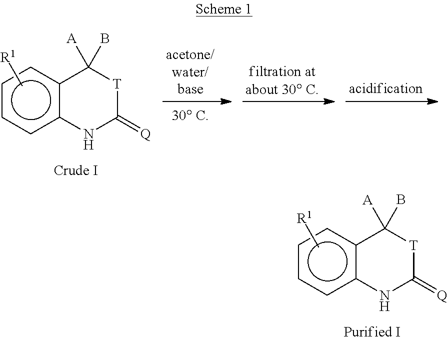

The crude compounds are first treated with a base in the presence of acetone and water to form a basic salt at a temperature of about 20 to about 60° C. In one embodiment, the temperature is about 20 to about 30° C. In another embodiment, the temperature is about 30° C. One of skill in the art would readily be able to select a suitable base based on its basicity and the compound being purified. A number of bases can be used and include hydroxides, among others. In one example, alkali metal hydroxides (abbreviated MOH below) may be used. In another example, alkaline earth metal hydroxide (abbreviated MOH₂ below) may be used. Hydroxides can include, without limitation, sodium hydroxide, potassium hydroxide, and calcium hydroxide. Desirably, the base is sodium hydroxide.

Suitably, the ratio of base to the crude compound is at least 1:1. The ratio of base to acetone is about 2 to about 5% by weight. In one example, the ratio of base to acetone is about 2.5% by weight. The ratio of base to water is about 5 to about 10% by weight. In one example, the ratio of base to water is about 8% by weight. However, the process is not so limited, with higher or lower amounts of base, acetone and water being readily determined and utilized by one of skill in the art.

The amount of water and acetone utilized depends upon the scale of the reaction, i. e., the amount of reagents utilized. One of skill in the art would readily be able to determine the amount of water and acetone required to perform the purification.

Advantageously, the processes herein avoid the requirement of the classical crystallization processes in the art that utilize solvents that utilize acetone-water, which have been found to result in low yield.

In one embodiment, the basic salt is of the following structure, wherein A, B, T, and R¹ are defined above and M is an alkali or alkaline earth metal:

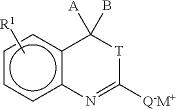

In another embodiment, the basic salt is of the following structure:

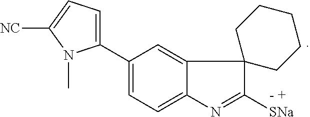

After formation of the basic salt, the base/acetone/water solution is filtered. The temperature of the filtration is desirably maintained at about 0 to about 60° C. In one embodiment, the temperature is about 20 to about 30° C. In another embodiment, the temperature is about 30° C. One of skill in the art would readily be able to select a suitable filtration unit. In one embodiment, the filtration is a Pall Gelman filtration unit. In another embodiment, the basic salt solution is filtered using a filtration unit containing charcoal, a filter aid such as diatomaceous earth (e.g., Celite™), or a combination thereof. In a further embodiment, the basic salt solution is filtered using a filtration unit containing charcoal and diatomaceous earth. In still another embodiment, the basic salt solution is filtered using a filtration unit containing alternating layers of charcoal and diatomaceous earth. In yet a further embodiment, the basic salt solution is filtered using a filtration unit containing a first layer of diatomaceous earth, a second layer of charcoal, and a third layer of diatomaceous earth. The inventors found that maintaining the filtration at a temperature of about 30° C. maximized absorption of color, impurities, and odor by the charcoal and thereby prevented premature crystallization of the purified product during filtration.

Once filtered, the basic salt is converted to the purified compound. Methods for converting the basic salt to the purified compound include acidification of the filtered base/acetone/water solution. The acidification may be performed using a variety of acids known in the art. In one embodiment, the acidification is performed by acidifying the solution to an acidic pH. In one example, the filtered solution is acidified to a pH of 0 to less than 7. In another example, the filtered solution is acidified to a pH of about 1 to 7. In a further example, the filtered solution is acidified to a pH of about 4.5 to less than about 7. In yet another example, the filtered solution is acidified to a pH of about 1.5. After acidification, the purified compound desirably precipitates in the solution. In one embodiment, the acidification is performed using hydrochloric acid.

After conversion to the purified compound, the precipitated purified compound can be isolated using techniques known to those of skill in the art and include filtration and centrifugation, among others. Desirably, the precipitated purified compound is collected by filtration. More desirably, the precipitated purified compound is collected by filtration at a temperature below the boiling point of the solvent, e.g., 1 to 35° C., 15° C. to 25° C., or about 20° C.

The purified compound can then be further purified using techniques known to those of skill in the art and include chromatography, distillation, drying, recrystallization, or combinations thereof. In one embodiment, the purified compound can be recrystallized from one or more solvents using techniques known to those of skill in the art. In a further embodiment, the purified compound is dissolved in a minimal amount of a solvent, the volume of the solution concentrated by removing some of the solvent, and the temperature of the solution cooled to promote precipitation of the twice-purified compound. One of skill in the art would readily be able to determine the amount of solvent required to recrystallize the purified compound. The twice-purified precipitated compound can then be isolated using techniques as previously discussed.

In another embodiment, the purified compound can be dried at atmospheric pressure or under a vacuum. One of skill in the art would readily be able to select a suitable vacuum to dry the purified compounds. Higher temperatures can also be applied to the purified compound during drying to remove entrapped solvents. Such temperatures can readily be selected by one of skill in the art.

The process desirably permits isolation of the purified progesterone receptor modulators in a yield greater than about 90%, about 91%, about 92%, about 93%, about 94%, about 95%, about 96%, about 97%, about 98%, and about 99%.

It is also desirable that the purified progesterone receptor modulator contains very few impurities. Impurities that are desirably absent from the purified progesterone receptor modulator include acetone, water, and any other remaining impurities. For example, the purified compound desirably contains less than 3% of impurities, less than 2% impurities, and less than 1% impurities. In one embodiment, the purified compound contains less than about 1% acetone. In another embodiment, the purified compound contains less than about 1.5% water.

The processes are typically performed under inert conditions including performing the process under an atmosphere of substantially nitrogen or argon. Further, the processes can be performed on laboratory scales or under scaled-up conditions such as on an at least one kilogram scale using the teachings provided herein.

In one embodiment, a process is provided for purifying a compound of the structure:

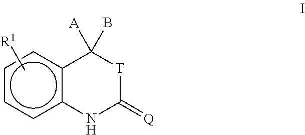

wherein, A and B are independently selected from among H, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₃ to C₈ cycloalkyl, substituted C₃ to C₈ cycloalkyl, aryl, substituted aryl, heterocyclic, substituted heterocyclic, COR^{A}, and NR^{B}COR^{A}; or A and B are joined to form a ring including (i), (ii), or (iii): (i) a carbon-based 3 to 8 membered saturated spirocyclic ring; (ii) a carbon-based 3 to 8 membered spirocyclic ring containing in its backbone one or more carbon-carbon double bonds; or (iii) a 3 to 8 membered heterocyclic ring containing in its backbone one to three heteroatoms selected from among O, S and N; the rings of (i), (ii) and (iii) being optionally substituted by from 1 to 4 groups selected from among fluorine, C₁ to C₆ alkyl, C₁ to C₆ alkoxy, C₁ to C₆ thioalkyl, CF₃, OH, CN, NH₂, NH(C₁ to C₆ alkyl), and N(C₁ to C₆ alkyl)₂; R^{A} is selected from among H, C₁ to C₃ alkyl, substituted C₁ to C₃ alkyl, aryl, substituted aryl, C₁ to C₃ alkoxy, substituted C₁ to C₃ alkoxy, amino, C₁ to C₃ aminoalkyl, and substituted C₁ to C₃ aminoalkyl; R^{B} is H, C₁ to C₃ alkyl, or substituted C₁ to C₃ alkyl; T is O, S, or absent; Q is O, S, or NR³; R¹ is (iv), (v), or (vi): (iv) halogen; (v) a substituted benzene ring containing the substituents X, Y and Z as shown below:

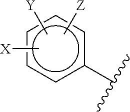

wherein, X is selected from among H, halogen, CN, C₁ to C₃ alkyl, substituted C₁ to C₃ alkyl, alkenyl, substituted alkenyl, alkynyl, substituted alkynyl, C₁ to C₃ alkoxy, substituted C₁ to C₃ alkoxy, C₁ to C₃ thioalkoxy, substituted C₁ to C₃ thioalkoxy, amino, C₁ to C₃ aminoalkyl, substituted C₁ to C₃ aminoalkyl, NO₂, C₁ to C₃ perfluoroalkyl, 5 or 6 membered heterocyclic ring containing in its backbone 1 to 3 heteroatoms, SO₂NH₂, COR^{C}, OCOR^{C}, and NR^{D}COR^{C}; R^{C} is H, C₁ to C₃ alkyl, substituted C₁ to C₃ alkyl, aryl, substituted aryl, C₁ to C₃ alkoxy, substituted C₁ to C₃ alkoxy, C₁ to C₃ aminoalkyl, or substituted C₁ to C₃ aminoalkyl; R^{D} is H, C₁ to C₃ alkyl, or substituted C₁ to C₃ alkyl; Y and Z are independently selected from among H, halogen, CN, NO₂, amino, aminoalkyl, C₁ to C₃ alkoxy, C₁ to C₃ alkyl, and C₁ to C₃ thioalkoxy; or (vi) a five or six membered ring having in its backbone 1, 2, or 3 heteroatoms selected from among O, S, SO, SO₂ and NR² and containing one or two substituents independently selected from among H, halogen, CN, NO₂, amino, C₁ to C₃ alkyl, C₁ to C₃ alkoxy, C₁ to C₃ aminoalkyl, SO₂NH₂, COR^{E}, and NR^{F}COR^{E}; R^{E} is H, C₁ to C₃ alkyl, substituted C₁ to C₃ alkyl, aryl, substituted aryl, C₁ to C₃ alkoxy, substituted C₁ to C₃ alkoxy, C₁ to C₃ aminoalkyl, or substituted C₁ to C₃ aminoalkyl; R^{F} is H, C₁ to C₃ alkyl, or substituted C₁ to C₃ alkyl; R² is H, absent, O, or C₁ to C₄ alkyl; and R³ is C₁ to C₆ alkyl, substituted C₁ to C₆ alkyl, aryl, substituted aryl, CN, C(O)R⁴, SO₂R⁴, SCN, OR⁴, SR⁴, C(O)OR⁴, C(S)OR⁴, C(O)SR⁴, or C(S)SR⁴; R⁴ is C₁ to C₆ alkyl, substituted C₁ to C₆ alkyl, aryl, or substituted aryl; wherein the process includes dissolving the compound of formula I in a solution containing acetone, water and a base at about 30° C.; (b) filtering the solution at a temperature of about 20° C. or higher; precipitating the purified compound of formula I by adjusting the filtered solution to an acidic pH. The inventors found that filtration at lower temperatures results in undesirably high levels of residual solvent. Therefore, temperatures above about 20° C., e.g., about room temperature, to about 30° C. were utilized. Optionally, higher temperatures may be selected for one of these steps (e.g., 35 to 40° C.).

In another embodiment, a process is provided for purifying 5-(2-thioxospiro[cyclohexane-1,3-[3H]indol]-5-yl)-1-methylpyrrole-2-carbonitrile, including dissolving 5-(2-thioxospiro[cyclohexane-1,3-[3H]indol]-5-yl)-1-methylpyrrole-2-carbonitrile in a solution containing acetone, water and a base at about 30° C.; filtering the solution at about 30° C.; and precipitating the purified 5-(2-thioxospiro[cyclohexane-1,3-[3H]indol]-5-yl)-1-methylpyrrole-2-carbonitrile by adjusting the filtered solution to an acidic pH. See, Scheme 2.

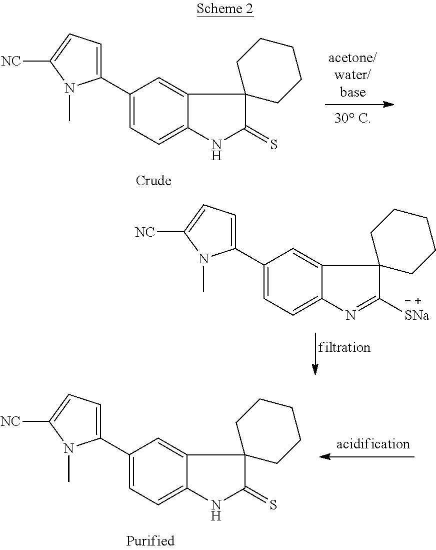

In another embodiment, a process is provided for purifying 5-(2-thioxospiro[cyclohexane-1,3-[3H]indol]-5-yl)-1-methylpyrrole-2-carbonitrile, including (a) dissolving 5-(2-Thioxospiro[cyclohexane-1,3-[3H]indol]-5-yl)-1-methylpyrrole-2-carbonitrile is acetone, water and sodium hydroxide at about 30° C.; (b) filtering the solution of step (a) through charcoal at about 30° C.; (c) precipitating the purified 5-(2-thioxospiro[cyclohexane-1,3-[3H]indol]-5-yl)-1-methylpyrrole-2-carbonitrile by adjusting the solution of step (b) to a pH of about 4.5 to about 7 using hydrochloric acid; and (d) collecting the purified 5-(2-thioxospiro[cyclohexane-1,3-[3H]indol]-5-yl)-1-methylpyrrole-2-carbonitrile by filtration at about 20° C.

In still a further embodiment, a process is provided for purifying 5-(2-thioxospiro[cyclohexane-1,3-[3H]indol]-5-yl)-1-methylpyrrole-2-carbonitrile, including (a) dissolving 5-(2-thioxospiro[cyclohexane-1,3-[3H]indol]-5-yl)-1-methylpyrrole-2-carbonitrile in a solution containing acetone, water and sodium hydroxide at about 30° C.; (b) filtering the solution of step (a) at about 30° C.; and (c) precipitating the purified 5-(2-Thioxospiro[cyclohexane-1,3-[3H]indol]-5-yl)-1-methylpyrrole-2-carbonitrile by adjusting the solution of step (b) to a pH of about 4.5 to about 7; wherein the process is performed in the absence of acetone distillation.

In yet another embodiment, a process is provided for purifying 5-(2-thioxospiro[cyclohexane-1,3-[3H]indol]-5-yl)-1-methylpyrrole-2-carbonitrile, including (a) dissolving 5-(2-thioxospiro[cyclohexane-1,3-[3H]indol]-5-yl)-1-methylpyrrole-2-carbonitrile in a solution containing acetone, water and sodium hydroxide at about 30° C.; (b) filtering the solution of step (a) at about 30° C.; and (c) precipitating purified 5-(2-thioxospiro[cyclohexane-1,3-[3H]indol]-5-yl)-1-methylpyrrole-2-carbonitrile by adjusting the solution of step (b) to a pH of about 4.5 to about 7; wherein the process is performed in the absence of THF.

### III. Methods of Using the Purified Compounds

The purified compounds prepared herein are useful as progesterone receptor modulators, including antagonists and agonists. In one embodiment, the purified compounds can act as competitive inhibitors of progesterone binding to the PR and therefore act as agonists in functional models, either/or in vitro and in vivo.

The purified compounds are therefore useful as oral contraceptives in both males and females. The purified compounds are also useful in hormone replacement therapy. The purified compounds are further useful in the treatment of endometriosis, luteal phase defects, hormone-dependent neoplastic disease, benign breast and prostatic diseases, cycle-related symptoms, fibroids, leiomyomata, dysmenorrheal, dysfunctional uterine bleeding, symptoms of premenstrual syndrome and premenstrual dysphoric disorder; for inducing amenorrhea; and in the synchronization of estrus. The hormone-dependent neoplastic disease can include uterine myometrial fibroids, endometriosis, benign prostatic hypertrophy, carcinomas and adenocarcinomas of the endometrium, ovary, breast, colon, prostate, pituitary, uterine, and meningioma. The purified compounds are also useful in treating hirsutism or acne.

The term “cycle-related symptoms” refers to psychological symptoms (e.g., mood change, irritability, anxiety, lack of concentration, or decrease in sexual desire) and physical symptoms (e.g., dysmenorrhea, breast tenderness, bloating, fatigue, or food cravings) associated with a woman's menstrual cycle. Cycle-related symptoms include, but are not limited to, dysmenorrhea and moderate to severe cycle-related symptoms.

In one embodiment, the purified compounds are used alone as a sole therapeutic agent. In other embodiments, the purified compounds are used in combination with other agents, such as estrogens, progestins, estrones, or androgens.

The purified compounds encompass tautomeric forms of the structures provided herein characterized by the bioactivity of the drawn structures. Further, the purified compounds can be used in the form of pharmaceutically acceptable salts derived from pharmaceutically or physiologically acceptable acids, bases, alkali metals and alkaline earth metals.

Physiologically acceptable acids include those derived from inorganic and organic acids. A number of inorganic acids are known in the art and include hydrochloric, hydrobromic, hydroiodic, sulfuric, nitric, and phosphoric acids, among others. Similarly, a variety of organic acids are known in the art and include, without limitation, lactic, formic, acetic, fumaric, citric, propionic, oxalic, succinic, glycolic, glucuronic, maleic, furoic, glutamic, benzoic, anthranilic, salicylic, tartaric, malonic, mallic, phenylacetic, mandelic, embonic, methanesulfonic, ethanesulfonic, panthenoic, benzenesulfonic, toluenesulfonic, stearic, sulfanilic, alginic, and galacturonic acids, among others.

Physiologically acceptable bases include those derived from inorganic and organic bases. A number of inorganic bases are known in the art and include aluminium, calcium, lithium, magnesium, potassium, sodium, and zinc sulfate or phosphate compounds, among others. A number of organic bases are known in the art and include, without limitation, N,N,-dibenzylethylenediamine, chloroprocaine, choline, diethanolamine, ethylenediamine, meglumine, and procaine, among others.

Physiologically acceptable alkali salts and alkaline earth metal salts can include, without limitation, sodium, potassium, calcium and magnesium salts in the form of esters, and carbamates. Other conventional “pro-drug” forms can also be utilized which, when delivered in such form, convert to the active moiety in vivo.

These salts, as well as other purified compounds can be in the form of esters, carbamates and other conventional “pro-drug” forms, which, when administered in such form, convert to the active moiety in vivo. In one embodiment, the prodrugs are esters. See, e.g., B. Testa and J. Caldwell, “Prodrugs Revisited: The “Ad Hoc” Approach as a Complement to Ligand Design”, Medicinal Research Reviews, 16(3):233-241, ed., John Wiley & Sons (1996).

The purified compounds discussed herein also encompass “metabolites” which are unique products formed by processing the compounds by the cell or patient. In one embodiment, the metabolites are formed in vivo.

In one embodiment, the purified compounds are formulated neat. In other embodiments, the purified compounds are formulated with a pharmaceutical carrier for administration, the proportion of which is determined by the solubility and chemical nature of the compound, chosen route of administration and standard pharmacological practice. The pharmaceutical carrier may be solid or liquid.

A solid carrier can include one or more substances which may also act as flavoring agents, lubricants, solubilizers, suspending agents, fillers, glidants, compression aids, binders or tablet-disintegrating agents; it can also be an encapsulating material. In powders, the carrier is a finely divided solid which is in admixture with the finely divided active ingredient. In tablets, the active ingredient is mixed with a carrier having the necessary compression properties in suitable proportions and compacted in the shape and size desired. The powders and tablets may contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Liquid carriers are used in preparing solutions, suspensions, emulsions, syrups, elixirs and pressurized compositions. The purified compound can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators. Suitable examples of liquid carriers for oral and parenteral administration include water (partially containing additives as above, e.g. cellulose derivatives, such as sodium carboxymethyl cellulose solution), alcohols (including monohydric alcohols and polyhydric alcohols, e.g. glycols) and their derivatives, lethicins, and oils (e.g. fractionated coconut oil and arachis oil). For parenteral administration, the carrier can also be an oily ester such as ethyl oleate and isopropyl myristate. Sterile liquid carriers are useful in sterile liquid form compositions for parenteral administration. The liquid carrier for pressurized compositions can be halogenated hydrocarbon or other pharmaceutically acceptable propellant.

The purified compounds can be delivered by a route such as oral, dermal, transdermal, intrabronchial, intranasal, intravenous, intramuscular, subcutaneous, parenteral, intraperitoneal, intranasal, vaginal, rectal, sublingual, intracranial, epidural, intratracheal, or by sustained release. In one embodiment, delivery is oral or transdermal.

In another embodiment, the compositions are delivered orally by tablet, capsule, microcapsules, dispersible powder, granule, suspension, syrup, elixir, and aerosol. In one embodiment, when the compositions are delivered orally, delivery is by tablets and hard- or liquid-filled capsules.

In yet another embodiment, the purified compounds are delivered intravenously, intramuscularly, subcutaneously, parenterally and intraperitoneally in the form of sterile injectable solutions, suspensions, dispersions, and powders which are fluid to the extent that easy syringe ability exists. Such injectable compositions are sterile, stable under conditions of manufacture and storage, and free of the contaminating action of microorganisms such as bacteria and fungi.

Injectable formations can be prepared by combining the purified compounds with a liquid. The liquid can be selected from among water, glycerol, ethanol, propylene glycol and polyethylene glycol, oils, and mixtures thereof. In one embodiment, the liquid carrier is water. In another embodiment, the oil is vegetable oil. Optionally, the liquid carrier contains a suspending agent. In another embodiment, the liquid carrier is an isotonic medium and contains about 0.05 to about 5% suspending agent.

In a further embodiment, the purified compounds are delivered rectally in the form of a conventional suppository.

In another embodiment, the purified compounds are delivered vaginally in the form of a conventional suppository, cream, gel, ring, or coated intrauterine device (IUD).

In yet another embodiment, the purified compounds are delivered intranasally or intrabronchially in the form of an aerosol.

In a further embodiment, the purified compounds are delivered transdermally or by sustained release through the use of a transdermal patch containing the purified compounds and an optional carrier that is inert to the compound(s), is nontoxic to the skin, and allows for delivery of the purified compound(s) for systemic absorption into the blood stream. Such a carrier can be a cream, ointment, paste, gel, or occlusive device. The creams and ointments can be viscous liquid or semisolid emulsions. Pastes can include absorptive powders dispersed in petroleum or hydrophilic petroleum. Further, a variety of occlusive devices can be utilized to release the active reagents into the blood stream and include semi-permeable membranes covering a reservoir contain the active agents, or a matrix containing the active agents.

In one embodiment, sustained delivery devices are utilized in order to avoid the necessity for the patient to take medications on a daily basis. The term “sustained delivery” is used herein to refer to delaying the release of an active agent, i. e., the purified compounds, until after placement in a delivery environment, followed by a sustained release of the agent at a later time. A number of sustained delivery devices are known in the art and include hydrogels (U.S. Pat. Nos. 5,266,325; 4,959,217; 5,292,515), osmotic pumps (U.S. Pat. Nos. 4,295,987 and 5,273,752 and European Patent No. 314,206, among others); hydrophobic membrane materials, such as ethylenemethacrylate (EMA) and ethylenevinylacetate (EVA); bioresorbable polymer systems (International Patent Publication No. WO 98/44964 and U.S. Pat. Nos. 5,756,127 and 5,854,388); and other bioresorbable implant devices composed of, for example, polyesters, polyanhydrides, or lactic acid/glycolic acid copolymers (U.S. Pat. No. 5,817,343). For use in such sustained delivery devices, the purified compounds can be formulated as described herein. See, U.S. Pat. Nos. 3,845,770; 3,916,899; 3,536,809; 3,598,123; and 4,008,719.

The dosage requirements vary with the particular purified compounds utilized, compositions employed, the route of administration, the severity of the symptoms presented and the particular subject being treated. Based on the results obtained in the standard pharmacological test procedures, projected daily dosages of purified compound would be about 0.1 to about 500 mg/kg. In one example, the dosage of purified compound is about 1 to about 100 mg/kg. In another example, the dosage of purified compound is about 2 to about 80 mg/kg. In a further example, the dosage of purified compound is about 5 to about 50 mg/kg. In still another example, the dosage of purified compound is about 5 to about 25 mg/kg. Treatment will generally be initiated with small dosages less than the optimum dose of the purified compound. Thereafter, the dosage may be increased until the optimum effect under the circumstances is reached.

Advantageously, particularly potent PR modulators (e.g., those of formula I) may be useful at the lower end of the dosage ranges provided herein. The dosage regimen may however be adjusted to provide the optimal therapeutic response. For example, several divided doses (e.g., in divided doses 2 to 4 times a day) may be administered daily or the dose may be proportionally reduced as indicated by the exigencies of the therapeutic situation. Alternatively, a single dose can be delivered. In certain embodiments, the delivery can be on a daily, weekly, or monthly basis. In one embodiment, delivery is on a daily basis. Daily dosages can be lowered or raised based on the periodic delivery.

Precise dosages for oral, parenteral, nasal, or intrabronchial administration can be determined by the administering physician based on experience with the individual subject treated. In one embodiment, the pharmaceutical composition is in unit dosage form, e.g. as tablets or capsules. In such form, the composition is sub-divided in unit dose containing appropriate quantities of the active ingredient; the unit dosage forms can be packaged compositions, for example, packaged powders, vials, ampoules, pre filled syringes or sachets containing liquids. The unit dosage form can be, for example, a capsule or tablet itself, or it can be the appropriate number of any such compositions in package form.

### IV. Pharmaceutical Kits

Kits or packages of pharmaceutical formulations including the purified compounds of formula I are also described herein. When the purified compounds of formula I are to be delivered continuously, a package or kit can include the purified compound in each tablet. When the purified compound is to be delivered with periodic discontinuation, a package or kit can include placebos on those days when the purified compound is not delivered.

In one embodiment, the kits are also organized to indicate a single oral formulation or combination of oral formulations to be taken on each day of the cycle. In a further embodiment, the kits include oral tablets to be taken on each of the days specified. In still another embodiment, one oral tablet will contain each of the combined daily dosages indicated.

Similarly, other kits of the type described above may be prepared in which a purified compound of formula I is delivered. In one embodiment, the daily dosage of the purified compound of formula I remain fixed in each particular phase in which it is delivered. In a further embodiment, the daily dose units described are to be delivered in the order described, with the first phase followed in order by the second and third phases. In yet another embodiment, the kits contain the placebo described for the final days of the cycle to help facilitate compliance with each regimen.

A number of packages or kits are known in the art for the use in dispensing pharmaceutical agents for oral use. In one embodiment, the package has indicators for each day, and may be a labeled blister package, dial dispenser package, or bottle.

The following examples are illustrative only and are not intended to be a limitation on the present invention.

### EXAMPLES

### Example 1

### Purification of 5-(2-Thioxospiro[cyclohexane-1,3-[3H]indol]-5-yl)-1-methylpyrrole-2-carbonitrile

A. Sodium Salt Formation

Crude 5-(2-Thioxospiro[cyclohexane-1,3-[3H]indol]-5-yl)-1-methylpyrrole-2-carbonitrile (3.008 kg, 9.358 mol) was added to a solution of acetone (19 kg) and water (5.1 L) at 5° C. The temperature of the mixture was adjusted to 24° C. and a 50% sodium hydroxide solution was added (1.0 kg, 12.5 mol) by maintaining the temperature at 24 to 25° C. The caustic charge line was chased with 1 kg of water. The temperature of the solution was adjusted to 32° C. and stirred for 20 minutes to dissolve the solids.

B. Clarification

A Sparkler® filter (8-inch diameter) equipped with a basket and a polypropylene filter cloth folded inside was coated with a layer of the Celite™ 503 reagent (0.223 kg), followed by a layer of activated carbon (the Darco™ G-60 reagent, 0.306 kg), followed by another layer of the Celite™ 503 reagent (0.217 kg) to cover the charcoal. The above-noted sodium salt solution was circulated through the Sparkler® filter for about 30 minutes, transferred to a 10μ Polypure® DCF cartridge pre-filter, and then to a 0.2μ Polypure® DCF cartridge filter and collected. An acetone wash (1.2 kg) was passed through the three filters and combined with the filtered solution. The clarification was completed in about 1.2 hours.

C. Acidification and Precipitation

A mixture of concentrated HCl (1.4 kg) and water (12 kg) was added to the filtered solution of the sodium salt (11.3 kg, about 12 mol), which was at a temperature of about 33° C., through a 10μ, Polypure® DCF cartridge filter while maintaining a temperature of 33 to 36° C. over a period of about 1.4 hours. The pH of the solution was monitored and found to be 1.5.

The slurry was stirred at 34° C. for 31 minutes, cooled to 22° C. for at least 31 minutes, and held overnight for 17.8 hours at 21 to 22° C. for convenience. However, this is not a limitation, as the slurry can be filtered off immediately after stirring at about 25 to 25° C. for about 30 minutes.

D. Isolation and Drying of 5-(2-Thioxospiro[cyclohexane-1,3-[3H]indol]-5-yl)-1-methylpyrrole-2-carbonitrile

The slurry from the previous step was added to a 12-inch diameter Nutsche filter with a polypropylene cloth in two portions and the mother liquor was collected in a drum for a period of about one minute. The cake collected on the filter was washed at 20 to 25° C. with a solution of acetone (2.6 kg) and water (6.7 kg), the acetone/water solution being first passed through a 10μ filter. The washed cake was further washed with another solution of acetone (0.4 kg) and water (9.5 kg), the acetone/water solution again being first filtered through a 10μ filter.

The cake was dried on the Nutsche filter using nitrogen over a period of about 97 hours. The filter cake was dried further in the vacuum oven at 66° C. to a constant weight. The weight loss overnight after 17.7 hours was just 0.1%, indicating a dry cake.

Analyses for acetone and water content were 1.0% and 0.07% by weight, respectively, which met the target levels of not more than 1% acetone and 1.5% water. The final dry cake weight was 2.737 kg (91.0%).

The pre-milled dry 5-(2-Thioxospiro[cyclohexane-1,3-[3H]indol]-5-yl)-1-methylpyrrole-2-carbonitrile was analyzed by HPLC at a purity of 99.0 area % and at a strength of 98.6 weight %; particle size analysis gave a distribution of 90% less than 61.6 microns, 50% equal to 24.8μ and 10% less than 2.9μ.

All publications cited in this specification are incorporated herein by reference. While the invention has been described with reference to particular embodiments, it will be appreciated that modifications can be made without departing from the spirit of the invention. Such modifications are intended to fall within the scope of the appended claims.

1. A process for purifying a compound of the structure: 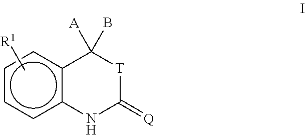
wherein:
A and B are independently selected from the group consisting of H, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₃ to C₈ cycloalkyl, substituted C₃ to C₈ cycloalkyl, aryl, substituted aryl, heterocyclic, substituted heterocyclic, COR^{A}, and NR^{B}COR^{A}; or
A and B are joined to form a ring comprising (i), (ii), or (iii):
(i) a carbon-based 3 to 8 membered saturated spirocyclic ring;
(ii) a carbon-based 3 to 8 membered spirocyclic ring containing in its backbone one or more carbon-carbon double bonds; or
(iii) a 3 to 8 membered heterocyclic ring containing in its backbone one to three heteroatoms selected from the group consisting of O, S and N;
the rings of (i), (ii) and (iii) being optionally substituted by from 1 to 4 groups selected from the group consisting of fluorine, C₁ to C₆ alkyl, C₁ to C₆ alkoxy, C₁ to C₆ thioalkyl, CF₃, OH, CN, NH₂, NH(C₁ to C₆ alkyl), and N(C₁ to C₆ alkyl)₂;
R^{A} is selected from the group consisting of H, C₁ to C₃ alkyl, substituted C₁ to C₃ alkyl, aryl, substituted aryl, C₁ to C₃ alkoxy, substituted C₁ to C₃ alkoxy, amino, C₁ to C₃ aminoalkyl, and substituted C₁ to C₃ aminoalkyl;
R^{B} is H, C₁ to C₃ alkyl, or substituted C₁ to C₃ alkyl;
T is O, S, or absent;
Q is O, S, or NR³;
R¹ is (iv), (v), or (vi):
(iv) halogen;
(v) a substituted benzene ring containing the substituents X, Y and Z as shown below: 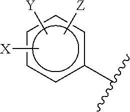
wherein:
X is selected from the group consisting of H, halogen, CN, C₁ to C₃ alkyl, substituted C₁ to C₃ alkyl, alkenyl, substituted alkenyl, alkynyl, substituted alkynyl, C₁ to C₃ alkoxy, substituted C₁ to C₃ alkoxy, C₁ to C₃ thioalkoxy, substituted C₁ to C₃ thioalkoxy, amino, C₁ to C₃ aminoalkyl, substituted C₁ to C₃ aminoalkyl, NO₂, C₁ to C₃ perfluoroalkyl, 5 or 6 membered heterocyclic ring containing in its backbone 1 to 3 heteroatoms, SO₂NH₂, COR^{C}, OCOR^{C}, and NR^{D}COR^{C};
R^{C} is H, C₁ to C₃ alkyl, substituted C₁ to C₃ alkyl, aryl, substituted aryl,
C₁ to C₃ alkoxy, substituted C₁ to C₃ alkoxy, C₁ to C₃ aminoalkyl, or substituted C₁ to C₃ aminoalkyl;
R^{D} is H, C₁ to C₃ alkyl, or substituted C₁ to C₃ alkyl;
Y and Z are independently selected from the group consisting of H, halogen, CN, NO₂, amino, aminoalkyl, C₁ to C₃ alkoxy, C₁ to C₃ alkyl, and C₁ to C₃ thioalkoxy; or
(vi) a five or six membered ring having in its backbone 1, 2, or 3 heteroatoms selected from the group consisting of O, S, SO, SO₂ and NR² and containing one or two substituents independently selected from the group consisting of H, halogen, CN, NO₂, amino, C₁ to C₃ alkyl, C₁ to C₃ alkoxy, C₁ to C₃ aminoalkyl, SO₂NH₂, COR^{E}, and NR^{E}COR^{E};
R^{E} is H, C₁ to C₃ alkyl, substituted C₁ to C₃ alkyl, aryl, substituted aryl, C₁ to C₃ alkoxy, substituted C₁ to C₃ alkoxy, C₁ to C₃ aminoalkyl, or substituted C₁ to C₃ aminoalkyl;
R^{E} is H, C₁ to C₃ alkyl, or substituted C₁ to C₃ alkyl;
R² is H, absent, O, or C₁ to C₄ alkyl; and
R³ is C₁ to C₆ alkyl, substituted C₁ to C₆ alkyl, aryl, substituted aryl, CN, C(O)R⁴, SO₂R⁴, SCN, OR⁴, SR⁴, C(O)OR⁴, C(S)OR⁴, C(O)SR⁴, or C(S)SR⁴;
R⁴ is C₁ to C₆ alkyl, substituted C₁ to C₆ alkyl, aryl, or substituted aryl;
wherein said process comprises:
(a) dissolving said compound of formula I in a solution comprising acetone, water and a base at about 30° C.;
(b) filtering the solution of step (a) at about 30° C.;
(c) precipitating said purified compound of formula I by adjusting the solution of step (b) to an acidic pH.

2. A process for purifying 5-(2-thioxospiro[cyclohexane-1,3-[3H]indol]-5-yl)-1-methylpyrrole-2-carbonitrile, comprising:
(a) dissolving 5-(2-thioxospiro[cyclohexane-1,3-[3H]indol]-5-yl)-1-methylpyrrole-2-carbonitrile in a solution comprising acetone, water and a base at about 30° C.;
(b) filtering the solution of step (a) at about 30° C. for at least about 30 minutes, using a pressurized filtration unit comprising a first layer of diatomaceous earth, a second layer of activated charcoal and a third layer of diatomaceous earth;
(c) circulating the solution of step (b) through a 10μ cartridge pre-filter;
(d) circulating the solution of step (c) through a 0.2μ cartridge filter; and
(e) precipitating said purified 5-(2-thioxospiro[cyclohexane-1,3-[3H]indol]-5-yl)-1-methylpyrrole-2-carbonitrile by adjusting the solution of step (d) to an acidic pH;
wherein said process is performed on a kilogram scale.

3. The process according to claim 2, wherein said base is sodium hydroxide, potassium hydroxide, or calcium hydroxide.

4. The process according to claim 2, wherein said base is sodium hydroxide.

5. The process according to claim 2, wherein said acidic pH is about 4.5 to about 7.

6. The process according to claim 2, wherein said purified compound is collected by filtration.

7. The process according to claim 6, wherein said purified compound is collected at about 20° C.

8. The process according to claim 2, which is performed under inert conditions.

9. The process according to claim 2, wherein said precipitation is performed using hydrochloric acid.

10. The process according to claim 2, wherein the product of step (a) is a sodium salt.

11. The process according to claim 2, wherein the product of step (a) is:

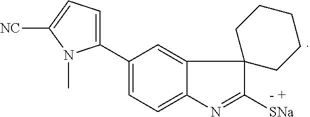

12. The process according to claim 2, wherein said purified compound is isolated in greater than an about 90% yield.

13. The process according to claim 2, wherein said purified compound contains less than about 1% acetone.

14. The process according to claim 2, wherein said purified compound contains less than about 1.5% water.

15. The process according to claim 2, wherein said acidic pH is about 1.5.

16. A process for purifying 5-(2-thioxospiro[cyclohexane-1,3-[3H]indol]-5-yl)-1-methylpyrrole-2-carbonitrile, comprising:
(a) dissolving 5-(2-thioxospiro[cyclohexane-1,3-[3H]indol]-5-yl)-1-methylpyrrole-2-carbonitrile in acetone, water and sodium hydroxide at about 30° C.;
(b) filtering the solution of step (a) through a pressurized filtration unit comprising a first layer of diatomaceous earth, a second layer of activated charcoal, and a third layer of diatomaceous earth for about 30 minutes at about 30° C.;
(c) circulating the solution of step (b) through a 10μ cartridge pre-filter;
(d) circulating the solution of step (c) through a 0.2μ cartridge filter;
(e) precipitating the purified 5-(2-thioxospiro[cyclohexane-1,3-[3H]indol]-5-yl)-1-methylpyrrole-2-carbonitrile by adjusting the solution of step (d) to a pH of about 4.5 to about 7 using hydrochloric acid; and
(f) collecting purified 5-(2-thioxospiro[cyclohexane-1,3-[3H]indol]-5-yl)-1-methylpyrrole-2-carbonitrile by filtration at about 20° C.;
wherein said process is performed on a kilogram scale.

17. A process for purifying 5-(2-thioxospiro[cyclohexane-1,3-[3H]indol]-5-yl)-1-methylpyrrole-2-carbonitrile, comprising:
(a) dissolving 5-(2-thioxospiro[cyclohexane-1,3-[3H]indol]-5-yl)-1-methylpyrrole-2-carbonitrile in a solution comprising acetone, water and sodium hydroxide at about 30° C.;
(b) filtering the solution of step (a) at about 30° C. for at least about 30 minutes, using a pressurized filtration unit comprising a first layer of diatomaceous earth, a second layer of activated charcoal, a third layer of diatomaceous earth;
(c) circulating the solution of step (b) through a 10μ cartridge pre-filter;
(d) circulating the solution of step (c) through a 0.2μ cartridge filter; and
(e) precipitating purified 5-(2-thioxospiro[cyclohexane-1,3-[3H]indol]-5-yl)-1-methylpyrrole-2-carbonitrile by adjusting the solution of step (d) to a pH of about 4.5 to about 7;
wherein said process is performed in the absence of acetone distillation and wherein on a kilogram scale.

18. A process for purifying 5-(2-thioxospiro[cyclohexane-1,3-[3H]indol]-5-yl)-1-methylpyrrole-2-carbonitrile, comprising:
(a) dissolving 5-(2-thioxospiro[cyclohexane-1,3-[3H]indol]-5-yl)-1-methylpyrrole-2-carbonitrile in a solution comprising acetone, water and sodium hydroxide at about 30° C.;
(b) filtering the solution of step (a) at about 30° C. for at least about 30 minutes, using a pressurized filtration unit comprising a first layer of diatomaceous earth, a second layer of activated charcoal, and a third layer of diatomaceous earth;
(c) circulating the solution of step (b) through a 10μ cartridge pre-filter;
(d) circulating the solution of step (c) through a 0.2μ cartridge filter; and
(e) precipitating purified 5-(2-Thioxospiro[cyclohexane-1,3-[3H]indol]-5-yl)-1-methylpyrrole-2-carbonitrile by adjusting the solution of step (d) to a pH of about 4.5 to about 7;
wherein said process is performed in the absence of THF and on a kilogram scale.
